(19) United States
(12) Patent Application Publication
(10) Pub. No.: US 2009/0004278 A1
(43) Pub. Date: Jan. 1, 2009

(54) Enzymatically Crosslinked Protein Nanoparticles

(75) Inventors: Makiko Aimi, Kanagawa (JP); Ryoichi Nemori, Kanagawa (JP); Yousuke Miyashita, Kanagawa (JP); Hiroshi Yokoyama, Kanagawa (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM CORPORATION, TOKYO (JP)

(21) Appl. No.: 12/160,175

(22) PCT Filed: Jan. 30, 2007
(86) PCT No.: PCT/JP2007/051856

§ 371 Date: Jul. 7, 2008

(30) Foreign Application Priority Data
Jan. 30, 2006 (JP) 2006-020020
Dec. 4, 2006 (JP) 2006-326640

(51) Int. Cl. A61K 9/14; C07K 2/00; C07K 14/47; C12P 21/02; C07K 14/76; A61K 47/42

(52) U.S. Cl. 424/489; 530/300; 514/773; 530/362; 530/354; 435/68.1; 977/906

(57) ABSTRACT

It is an object of the present invention to provide highly safe nanoparticles made from highly biocompatible materials without the use of a surfactant or synthetic polymer. The present invention provides a protein nanoparticle which is obtained by enzymatic crosslinking during and/or after the formation of protein nanoparticle.

### TECHNICAL FIELD

The present invention relates to enzymatically crosslinked protein nanoparticles, a method for producing the same, and use of the same.

### BACKGROUND ART

In the field of biotechnology, extensive applications of fine particle materials have been expected. In particular, application of nanoparticle materials produced with developments in nanotechnology to biotechnological or medical practice has been actively studied in recent years, and various research accomplishments have been reported.

In the drug delivery system (DDS) field, use of nanoparticles has been expected since an early stage, and nanoparticles have been considered very promising as carriers of drug or gene. In particular, research using polymeric micelles has been actively carried out. In many cases, AB or ABA type block copolymers are employed because of their simple structures. Polymeric micelles are characterized by large drug loading capacity, high water solubility, high structural stability, nonaccumulativeness, small particle diameters (not greater than 100 nm), and functional separability. Thus, research aiming at the targeting of a target site or at the solubilization of hydrophobic drugs has been conducted.

At solid cancer lesions, vascular endothelial permeability is abnormally enhanced and, simultaneously, discharge by the lymphatic system is inhibited. Accordingly, polymers inherently tend to selectively remain at cancer sites. Such a trait is referred to as the EPR (enhanced permeability and retention) effect (see, for example, Y. Matsumura and H. Maeda, Cancer Res., 46, 6387, 1986). In order to exhibit this EPR effect, the optimal size of a polymer is between 5 nm and 200 nm, the surface thereof is required to be hydrophilic, and such surface is required to be neutrally or weakly negatively charged. The sizes of polymeric micelles are within this range, and a hydrophobic inner core comprising a hydrophobic drug sealed therein is surrounded by a hydrophilic outer envelope. Thus, the surface properties thereof are of hydrophilic and such properties fulfill the aforementioned conditions. Accordingly, polymeric micelles are carrier systems suitable for realizing the EPR effect.

Some of the recently developed highly active drugs, such as taxol, are insoluble in water. Anticancer drugs that are difficult to absorb orally are preferably administered directly into the blood. Accordingly, such water-insoluble drugs are rendered soluble in water with the use of an organic solvent or surfactant, although the toxicity level of such organic solvent or surfactant used can be considerable. In order to alleviate the shock diseases resulting from such toxicity, it is required to previously administer steroids or to deliver the agent into the heart with the aid of a catheter. This results in a form of therapy that requires hospitalization. An administration method wherein a drug is sealed in a micelle structure of an amphipathic polymer that is considered to be less toxic than an organic solvent or low-molecular-weight surfactant and the resultant is administered directly into the blood, has been studied, and clinical testing has been also carried out (see, for example, Y. Mizumura et al., Jap. J. Cancer Res., 93, 1237, 2002).

In recent years, the production of cosmetic products has employed various new techniques including nanotechnology in order to improve functionality and usability and to differentiate products of interest from products of other companies, and more apparent effects on the skin have become desired. In general, a keratin layer exists as a barrier on the skin and thus the ability of drugs to permeate the skin is poor. In order to fully exhibit effects on the skin, it is essential to improve the skin permeability of active ingredients. Even though some ingredients have high efficacy on the skin, some such ingredients are difficult to formulate due to poor storage stability or the likelihood of imposing stimuli on the skin. In order to overcome such drawbacks, development of a variety of capsules is attempted while aiming at the improvement of transdermal absorption and storage stability and at the reduction of skin stimulation. At present, various materials such as ultrafine emulsions or liposomes have been studied (see, for example, Mitsuhiro Nishida, Fragrance Journal, Nov. 17, 2005). However, the safety of surfactants used for emulsification is an issue of concern, and structure formation with the aid of an ion complex is poorer in stability, compared with a covalent bond.

With the use of polymeric materials, remarkable improvement can be expected in storage stability or particle stability in vivo. Most studies, however, involve the use of synthetic polymers resulting from emulsion polymerization or other procedures. Although toxicity is reduced compared with the use of low-molecular weight compounds, a certain degree of toxicity has to be anticipated. Thus, development of safer carriers has been awaited.

Naturally occurring polymers exhibit high structural stability equivalent to that of synthetic polymers, exhibit higher levels of safety than synthetic polymers, and are also advantageous as DDS carriers. Compared with synthetic polymers, it is more difficult to produce carrier particles of naturally occurring polymers. Examples of means for producing naturally occurring polymer particles include spray drying, lyophilization, and a jet mill. In most cases, the particles are in the micron size, and it is difficult to control the particle size.

JP Patent Publication (Kokai) No. 2002-308728 A suggests a transdermal absorbent using nanoparticles of polymer material. This absorbent is an emulsion using a surfactant, and safety and stability are issues of concern as described above. JP Patent Publication (Kohyo) No. 2005-500304 A discloses spherical protein particles, the particle size of which as drug-containing compositions is not smaller than 1 μm. The formation of such particle is realized with the aid of a precipitant only, and they do not exhibit protein-protein networks resulting from covalent bonding. Thus, such protein particles are disadvantageous in terms of storage stability and particle stability in vivo. JP Patent Publication (Kohyo) No. 2001-502721 A suggests a drug-targeting system utilizing nanoparticles prepared from polymeric materials (synthetic or naturally occurring polymers). This technique comprises a step of polymerizing at least one monomer and/or oligomer precursor as part of a method of particle preparation. Even if naturally occurring polymers are used as polymeric materials, accordingly, safety is still an issue of concern. JP Patent Publication (Kokai) No. 2004-244420 A also suggests crosslinked polymer nanoparticles comprising skin care ingredients. This technique also comprises a step of polymerizing monomers or macromers (synthetic polymers having polymerizable groups) and thus safety is still an issue of concern. In C. Coester et al., Journal of Microencapsulation, 17, 189, 2000, particles insolubilized with the addition of an organic solvent to an aqueous gelatin solution are crosslinked with the use of glutaraldehyde. Glutaraldehyde is a highly toxic material and safety is an issue of concern when it remains. As described above, production of conventional polymer nanoparticles from naturally occurring polymers as well as from synthetic polymers involves the use of a surfactant, polymerizable monomer, chemical crosslinking agent, or the like as part of the process of particle formation. Thus, a safety issue still remains.

In general, proteins are chemically crosslinked. For example, a method involving the addition of a crosslinking agent such as glutaraldehyde as described above, a method comprising UV application with the use of a monomer having a photoreactive group, and a method of causing crosslinking by locally generating radicals via pulse irradiation are known. In contrast, as a method that makes use of traits of biopolymers, a method wherein the acyl transfer reaction of a glutamine residue is catalyzed with the use of transglutaminase to form intercellular or intracellular crosslinking is available (see, for example, JP Patent Publication (Kokai) No. 64-27471 A (1989). This crosslinking usually takes place in bulk or hydrous biopolymers, and formation of crosslinking in protein nanoparticles has not previously been known. Further, crosslinking between nanoparticles dispersed in an organic solvent has not previously been known.

### DISCLOSURE OF THE INVENTION

It is an object of the present invention to solve the problems of the aforementioned conventional techniques. That is, it is an object of the present invention to provide highly safe nanoparticles made from highly biocompatible materials without the use of a surfactant or synthetic polymer. Further, it is an object of the present invention to provide highly safe nanoparticles which were crosslinked without the use of a synthetic chemical crosslinking agent.

The present inventors have conducted concentrated studies in order to attain the above objects. As a result, they have found that protein nanoparticles can be produced by performing enzymatic crosslinking during and/or after the formation of protein nanoparticles. The present invention has been completed based on such finding.

Specifically, the present invention provides a protein nanoparticle which is obtained by enzymatic crosslinking during and/or after the formation of protein nanoparticle.

Preferably, enzymatic crosslinking is performed with the addition of crosslinking enzymes in a weight that is 0.1% to 100% of the protein weight.

Preferably, an enzyme used for crosslinking is transglutaminase.

Preferably, enzymatic crosslinking is carried out in an organic solvent.

Preferably, the protein nanoparticle of the present invention further comprises at least one active ingredient.

Preferably, the protein nanoparticle of the present invention comprises the active ingredient in a weight that is 0.1% to 100% of the protein weight.

Preferably, the active ingredient is an ingredient for a cosmetic, functional food, or pharmaceutical product.

Preferably, the ingredient of a cosmetic product is a moisturizer, skin-whitening agent, hair restoration tonics, hormone drugs or antiaging agent, the ingredient of a functional food is a vitamin or antioxidant, and the ingredient of a pharmaceutical product is an anticancer agent, antiallergic agent, antithrombotic agent, immunosuppressive agent, therapeutic agent for skin diseases, antifungal agent, nucleic acid medication or antiinflammatory agent.

Preferably, the average particle size is between 10 nm and 1,000 nm.

Preferably, the protein has a lysine residue and a glutamine residue.

Preferably, the protein is at least one selected from the group consisting of collagen, gelatin, albumin, casein, transferrin, globulin, fibroin, fibrin, laminin, fibronectin, and vitronectin.

Preferably, the protein is one which is derived from bovine, swine or fish, or a recombinant protein.

Preferably, the protein is an acid-treated gelatin.

Preferably, a phospholipid is added in a weight that is 0.1% to 100% of the protein weight.

Preferably, a cationic or anionic polysaccharide is added in a weight that is 0.1% to 100% of the protein weight.

Preferably, a cationic or anionic protein is added in a weight that is 0.1% to 100% of the protein weight.

Another aspect of the present invention provides a drug delivery agent which comprises the protein nanoparticle of the present invention as mentioned above.

Preferably, the drug delivery agent is used as transdermal absorbents, topical therapeutic agents, oral therapeutic agents, intradermal injections, hypodermic injections, intramuscular injections, intravenous injections, cosmetic products, functional foods, or supplements.

Preferably, the drug delivery agent comprises an additive.

Preferably, the additive is at least one member selected from among moistening agents, softening agents, transdermal absorption promoters, soothing agents, antiseptic agents, antioxidants, pigments, thickeners, aroma chemicals, and pH adjusters.

Further another aspect of the present invention provides a method for producing a protein nanoparticle which comprise performing enzymatic crosslinking during and/or after the formation of protein nanoparticle.

Further another aspect of the present invention provides a method for producing a protein nanoparticle which comprises performing enzymatic crosslinking during and/or after the formation of protein nanoparticle in an organic solvent.

### BRIEF DESCRIPTION OF THE DRAWINGS

FIG. 1 shows the results of photographing the gelatin nanoparticles of the present invention.

FIG. 2 shows the results of the cytotoxicity test of an aqueous solution of adriamycin and of adriamycin-encapsulating gelatin nanoparticles.

### BEST MODE FOR CARRYING OUT THE INVENTION

Hereafter, the embodiments of the present invention are described in greater detail.

The protein nanoparticle of the present invention is obtained by performing enzymatic crosslinking during and/or after the formation of protein nanoparticle.

The protein nanoparticle of the present invention does not comprise magnetic responsive particles.

The type of protein used in the present invention is not particularly limited. A protein having a lysine residue and a glutamine residue is preferable, and use of a protein having a molecular weight of approximately 10,000 to 1,000,000 is preferable. The origin of the protein is not particularly limited, and use of a human-derived protein is preferable. Examples of the protein that can be used include one selected from the group consisting of collagen, gelatin, albumin, casein, transferring, globulin, fibroin, fibrin, laminin, fibronectin, and vitronectin. The origin of the protein is not particularly limited, and any one which is derived from bovine, swine or fish, or a recombinant protein can be used. For example, the recombinant gelatin described in EP 1014176A2 and U.S. Pat. No. 6,992,172 can be used, but the gelatin is not limited thereto. Among them, an acid-treated gelatin, collagen, and albumin are particularly preferable. Acid-treated gelatin is most preferable.

In the present invention, a single protein may be used, or combinations of two or more proteins may be used.

In the present invention, an enzyme is not particularly limited, provided that its activity of protein crosslinking is known. Transglutaminase is particularly preferable.

Transglutaminase may be derived from a mammalian animal or microorganism, or may be a recombinant protein. Specific examples thereof include Activa series (Ajinomoto Co.) and mammalian-derived transglutaminase sold as reagents, such as guinea pig liver transglutaminase, goat-derived transglutaminase, rabbit-derived transglutaminase, and human-derived transglutaminase (manufactured by Oriental Yeast Co., Ltd., Upstate USA Inc., and Biodesign International).

The amount of enzyme used in the present invention can be adequately determined in accordance with protein type. In general, enzyme can be added in weights that are approximately 0.1% to 100% of the protein weight, with approximately 1% to 50% being preferable.

The duration of enzymatic crosslinking reaction can be adequately determined in accordance with the protein type and the sizes of nanoparticles. Such reaction can be generally carried out from 1 hour to 72 hours, and preferably from 2 hours to 24 hours.

The temperature at which an enzymatic crosslinking reaction is carried out can be adequately determined in accordance with the protein type and the sizes of nanoparticles. Such reaction can be generally carried out at 0° C. to 80° C., and preferably at 25° C. to 60° C.

In the present invention, a single enzyme can be used, or combinations of two or more enzymes may be used.

The average particle size of the nanoparticles of the present invention is generally 1 to 1,000 nm, preferably 10 to 1,000 nm, more preferably 50 to 500 nm, and particularly preferably 100 to 500 nm. Because of such particle sizes on the nano-level, the nanoparticles of the present invention can reach at any extremely small sites, such as capillary blood vessels.

The protein nanoparticle of the present invention preferably comprises at least one active ingredient. The amount of the active ingredient is not particularly limited. In general, the nanoparticles can comprise the active ingredient in a weight that is 0.1% to 100% of the protein weight.

In the present invention, the active ingredient may be added during or after the formation of the protein nanoparticle.

The active ingredient used in the present invention is an ingredient of a cosmetic product, such as a moisturizer, skin-whitening agent, hair restoration tonics, hormone drugs, or antiaging agent, an ingredient of a functional food, such as a vitamin or antioxidant, and an ingredient of a pharmaceutical product, such as an anticancer agent, antiallergic agent, antithrombotic agent, immunosuppressive agent, therapeutic agent for skin diseases, antifungal agent, nucleic acid medication or antiinflammatory agent. Specific examples of moisturizing agents used in the present invention include, but are not limited to, hyaluronic acid, ceramide, Lipidure, isoflavone, amino acid, collagen, mucopolysaccharide, fucoidan, lactoferrin, sorbitol, chitin and chitosan, malic acid, glucuronic acid, placenta extract, seaweed extract, moutan bark extract, sweet hydrangea leaf extract, Hypericum extract, coleus extract, Euonymus japonica extract, safflower extract, Rosa rugosa flower extract, Polyporus Sclerotium extract, hawthorn extract, rosemary extract, duku extract, chamomile extract, lamium album extract, Litchi Chinensis extract, Achillea Millefolium extract, aloe extract, marronnier extract, Thujopsis dolabrata extract, Fucus extract, Osmoin extract, oat extract, Tuberosa polysaccharide, Cordyceps Sinensis extract, barley extract, orange extract, Rehmannia root extract, zanthoxylum fruit extract, and coix seed extract.

Specific examples of skin-whitening agents used in the present invention include, but are not limited to, vitamin C and a derivative thereof, arbutin, hydroquinone, kojic acid, Lucinol, ellagic acid, tranexamic acid, and glutathione.

Specific examples of hair restoration tonics that are used in the present invention include, but are not limited to, adenosine, cepharanthin, glycyrrhetic acid or a derivative thereof, glycyrrhizin acid or a derivative thereof, isopropyl methyl phenol, pantothenic acid, panthenol, t-flavanone, tocopherols or a derivative thereof, hinokitiol, pentadecanoic acid or a derivative thereof, licorice extract, Lepisorus extract, sophora root extract, swertia herb extract, capsicum extract, Ampelopsis cantoniensis var. grossedentata extract, carrot extract, Taraxacum extract, tree peony extract, orange extract, blood circulation promoters (e.g., nicotinic acid, benzyl nicotinate, tocopherol nicotinate, nicotinic acid β-butoxy ester, minoxidil or an analog thereof, swertia herb extract, γ-oxazole, alkoxycarbonylpyridine N-oxide, carpronium chloride, and acetylcholine or a derivative thereof), antiinflammatory agents, and moistening agents.

Specific examples of hormone drugs that are used in the present invention include, but are not limited to, estradiol, ethinyl estradiol, estron, cortisone, hydrocortisone, prednisone, and prednisolone.

Specific examples of antiaging agents used in the present invention include, but are not limited to, retinoic acid, retinol, vitamin C and a derivative thereof, kinetin, β-carotene, astaxanthin, and tretinoin.

Specific examples of vitamins that are used in the present invention include, but are not limited to, vitamin A and a derivative thereof, retinoic acid, vitamin B family (e.g., vitamin B1, vitamin B2, vitamin B6, vitamin B12, and folic acid), vitamin C and a derivative thereof, vitamin D, vitamin E, vitamin F, pantothenic acid, and vitamin H.

Specific examples of antioxidants used in the present invention include, but are not limited to, a vitamin C and a derivative thereof, vitamin E, kinetin, α-lipoic acid, coenzyme Q10, polyphenol, SOD and phytic acid.

Specific examples of anticancer agents used in the present invention include, but are not limited to: fluorinated pyrimidine antimetabolites (for example, 5-fluorouracil (5-FU), tegafur, doxifluridine, and capecitabine); antibiotics (for example, mitomycin (MMC) and adriacin (DXR)); purine antimetabolites (for example, folic acid antagonists such as methotrexate and mercaptopurine); active metabolites of vitamin A (for example, antimetabolites such as hydroxy carbamide, tretinoin, and tamibarotene); molecular targeting agents (for example,, Herceptin and imatinib mesylate); platinum agents (for example, Briplatin or Randa (CDDP), Paraplatin (CBDC), Elplat (Oxa), and Akupura); plant alkaloids (for example, Topotecin or Campto (CPT), taxol (PTX), Taxotere (DTX), and Etoposide); alkylating agents (for example, busulphan, cyclophosphamide, and ifomide); antiandrogenic agents (for example, bicalutamide and flutamide); estrogenic agents (for example, fosfestrol, chlormadinone acetate, and estramustine phosphate); LH-RH agents (for example, Leuplin and Zoladex); antiestrogenic agents (for example, tamoxifen citrate and toremifene citrate); aromatase inhibitors (for example, fadrozole hydrochloride, anastrozole, and exemestane); progestational agents (for example, medroxyprogesterone acetate); and BCG.

Specific examples of antiallergic agents used in the present invention include, but are not limited to: mediator antireleasers, such as disodium cromoglycate and tranilast; histamine H1 antagonists, such as ketotifen fumarate and azelastine hydrochloride; thromboxane inhibitors, such as ozagrel hydrochloride; leukotriene antagonists, such as pranlukast; and suplatast tosylate.

Specific examples of antithrombotic agents that are used in the present invention include, but are not limited to, aspirin, ticlopidine hydrochloride, cilostazol, and warfarin potassium.

Specific examples of immunosuppressive agents that are used in the present invention include, but are not limited to, rapamycin, tacrolimus, ciclosporin, prednisolone, methylprednisolone, mycophenolate mofetil, azathioprine, and mizoribine.

Specific examples of therapeutic agents for skin diseases that are used in the present invention include, but are not limited to: therapeutic agents for atopic dermatitis (e.g., steroids, such as hydrocortisone butyrate, clobetasone butyrate, alclometasone propionate, clobetasol propionate, betamethasone dipropionate, and difluprednate, immunosuppressive agents such as tacrolimus, nonsteroids, such as bufexamac, ufenamate, ibuprofen piconol, and bendazac, zinc oxide, azulene, diphenhydramine, crotamiton, and moistening agents); acne medications, such as sulfur, salicylic acid, resorcin, thioxolone, selenium sulfide, nadifloxacin, gentamicin sulfate, tetracycline hydrochloride, clindamycin phosphate, and retinoic acid; and therapeutic agents for eczema.

Specific examples of antifungal agents that are used in the present invention include, but are not limited to, clotrimazole, bifonazole, miconazole nitrate, econazole nitrate, sulconazole nitrate, neticonazole hydrochloride, cloconazole hydrochloride, lanoconazole, ketoconazole, luliconazole, amorolfine hydrochloride, terbinafine hydrochloride, and tolnaftate.

Specific examples of nucleic acid medications that are used in the present invention include, but are not limited to, antisense, ribozyme, siRNA, aptamer, and decoy nucleic acids.

Specific examples of an antiinflammatory agent that can be used in the present invention include, but are not limited to, a compound which is selected from azulene, allantoin, lysozyme chloride, guaiazulene, diphenhydramine hydrochloride, hydrocortisone acetate, prednisolone, glycyrrhizinic acid, glycyrrhetinic acid, glutathione, saponin, methyl salicylate, mefenamic acid, phenylbutazone, indometacin, ibuprofen and ketoprofen, and its derivative and its salt; and a plant extract which is selected from Scutellariae Radix extract, Artemisia capillaris Thunb. Extract, Platycodon grandiflorum extract, Armeniacae Semen extract, Common gardenia extract, Sasa veitchii extract, Gentiana lutea extract, Comfrey extract, white birch extract, Malva extract, Persicae Semen extract, peach blade extract, and loquat blade extract.

In the present invention, a single active ingredient may be used, or combinations of two or more active ingredients may be used.

The protein nanoparticle of the present invention can be produced in accordance with the methods disclosed in JP Patent Publication (Kokai) No. 6-79168 A (1994) or in C. Coester et al., Journal of Microencapsulation, 17, pp. 187-193, 2000. It should be noted that crosslinking involves the use of enzymes instead of glutaraldehyde.

In the present invention, enzymatic crosslinking is preferably carried out in an organic solvent. Examples of organic solvents that are preferably used in the present invention include water-soluble organic solvents, such as ethanol, isopropanol, acetone, and THF.

Specific examples of phopholipids that can be used in the present invention include, but are not limited to, phosphatidyl choline (lecithin), phosphatidylethanolamine, phosphatidylserine, phosphatidylinositol, phosphatidylglycerol, diphosphatidylglycerol, and sphingomyelin.

The term “anionic polysaccharides” that is used in the present invention refers to polysaccharides having acidic polar groups such as carboxyl, sulfate or phosphate groups. Specific examples thereof include, but are not limited to, chondroitin sulfate, dextran sulfate, carboxymethylcellulose, carboxymethyl dextran, alginic acid, pectin, carragheenan, fucoidan, agaropectin, porphyran, karaya gum, gellan gum, xanthan gum, and hyaluronic acids.

The term “cationic polysaccharides” used in the present invention refers to polysaccharides having basic polar groups such as amino groups. Specific examples thereof include, but are not limited to, polysaccharides comprising glucosamine or galactosamine as a constitutive monosaccharide such as chitin or chitosan.

The term “anionic proteins” used in the present invention refers to proteins and lipoproteins whose isoelectric points are more basic than the physiological pH. Specific examples thereof include, but are not limited to, polyglutamic acid, polyaspartic acid, lysozyme, cytochrome C, ribonuclease, trypsinogen, chymotrypsinogen, and α-chymotrypsin.

The term “cationic proteins” used in the present invention refers to proteins and lipoproteins whose isoelectric points are more acidic than the physiological pH. Specific examples thereof include, but are not limited to, polylysine, polyarginine, histone, protamine, and ovalbumin.

The protein nanoparticle of the present invention can comprise active ingredients, and such protein nanoparticle can be administered to affected areas. Specifically, the protein nanoparticle of the present invention is useful as drug delivery agent.

Preferably, the protein nanoparticle of the present invention is administered by, for example, transdermal or transmucosal absorption or injection into a blood vessel, body cavity or lymph. Transdermal or transmucosal absorption is more preferable.

In the present invention, applications of drug delivery agents are not particularly limited. Examples of such applications include transdermal absorbents, topical therapeutic agents, oral therapeutic agents, intradermal injections, hypodermic injections, intramuscular injections, intravenous injections, cosmetic products, and supplements.

In the present invention, the drug delivery agent can comprise an additive. Additives are not particularly limited, and examples thereof include moistening agents, softening agents, transdermal absorption promoters, soothing agents, antiseptic agents, antioxidants, pigments, thickeners, aroma chemicals, and pH adjusters.

Specific examples of moistening agents that can be used in the present invention include, but are not limited to, agar, diglycerine, distearyldimonium hectorite, butylene glycol, polyethylene glycol, propylene glycol, hexylene glycol, coix seed extract, vaseline, urea, hyaluronic acid, ceramide, Lipidure, isoflavone, amino acid, collagen, mucopolysaccharide, fucoidan, lactoferrin, sorbitol, chitin and chitosan, malic acid, glucuronic acid, placenta extract, seaweed extract, moutan bark extract, sweet hydrangea leaf extract, Hypericum extract, coleus extract, Euonymus japonica extract, safflower extract, Rosa rugosa flower extract, Polyporus Sclerotium extract, hawthorn extract, rosemary extract, duku extract, chamomile extract, lamium album extract, Litchi Chinensis extract, Achillea Millefolium extract, aloe extract, marronnier extract, Thujopsis dolabrata extract, Fucus extract, Osmoin extract, oat extract, Tuberosa polysaccharide, Cordyceps Sinensis extract, barley extract, orange extract, Rehmannia root extract, zanthoxylum fruit extract, and coix seed extract.

Specific examples of softening agents that can be used in the present invention include, but are not limited to, glycerine, mineral oil, and emollient ingredients such as isopropyl isostearate, polyglyceryl isostearate, isotridecyl isononanoate, octyl isononanoate, oleic acid, glyceryl oleate, cacao butter, cholesterol, mixed triglyceride, dioctyl succinate, sucrose tetrastearate triacetate, cyclopentasiloxane, sucrose distearate, octyl palmitate, octyl hydroxy stearate, alkyl behenate, sucrose polybehenate, polymethylsilsesquioxane, myristyl alcohol, cetyl myristate, myristyl myristate, and hexyl laurate.

Specific examples of transdermal absorption promoters that can be used in the present invention include, but are not limited to, ethanol, isopropyl myristate, citric acid, squalane, oleic acid, menthol, N-methyl-2-pyrrolidone, diethyl adipate, diisopropyl adipate, diethyl sebacate, diisopropyl sebacate, isopropyl palmitate, isopropyl oleate, octyl dodecyl oleate, isostearyl alcohol, 2-octyldodecanol, urea, vegetable oil, and animal oil.

Specific examples of soothing agents that can be used in the present invention include, but are not limited to, benzyl alcohol, procaine hydrochloride, xylocaine hydrochloride, and chlorobutanol.

Specific examples of antiseptic agents that can be used in the present invention include, but are not limited to, benzoic acid, sodium benzoate, paraben, ethylparaben, methylparaben, propylparaben, butylparaben, potassium sorbate, sodium sorbate, sorbic acid, sodium dehydroacetate, hydrogen peroxide, formic acid, ethyl formate, sodium hypochlorite, propionic acid, sodium propionate, calcium propionate, pectin digests, polylysine, phenol, isopropylmethylphenol, orthophenyl phenol, phenoxyethanol, resorcin, thymol, thiram, and tea tree oil.

Specific examples of antioxidants that can be used in the present invention include, but are not limited to, vitamin C and a derivative thereof, vitamin E, kinetin, polyphenol, SOD, phytic acid, BHT, BHA, propyl gallate, fullerene, and citric acid.

Specific examples of pigments that can be used in the present invention include, but are not limited to, krill pigment, orange pigment, cocoa pigment, kaolin, carmines, ultramarine blue, cochineal pigment, chromium oxide, iron oxide, titanium dioxide, coal-tar color, and chlorophyll.

Specific examples of thickeners that can be used in the present invention include, but are not limited to, quince seed, carragheenan, gum Arabic, karaya gum, xanthan gum, gellan gum, Tamarind gum, Locust bean gum, gum tragacanth, pectin, starch, cyclodextrin, methylcellulose, ethylcellulose, carboxymethylcellulose, sodium alginate, polyvinyl alcohol, polyvinylpyrrolidone, carboxyvinyl polymer, and sodium polyacrylate.

Specific examples of aroma chemicals that can be used in the present invention include, but are not limited to, musk, acacia oil, anise oil, ylang ylang oil, cinnamon oil, jasmine oil, sweet orange oil, spearmint oil, geranium oil, thyme oil, neroli oil, mentha oil, hinoki (Japanese cypress) oil, fennel oil, peppermint oil, bergamot oil, lime oil, lavender oil, lemon oil, lemongrass oil, rose oil, rosewood oil, anisaldehyde, Geraniol, citral, civetone, muscone, limonene, and vanillin.

Specific examples of pH adjusters that can be used in the present invention include, but are not limited to, sodium citrate, sodium acetate, sodium hydroxide, potassium hydroxide, phosphoric acid, and succinic acid.

The dosage of the protein nanoparticle of the present invention can be adequately determined in accordance with, for example, the types and amounts of active ingredients used and the body weight and pathological condition of a patient. In general, approximately 10 μg to 100 mg of the protein nanoparticle can be administered per kg of the patient's body weight in a single dose, and approximately 20 μg to 50 mg thereof can be preferably administered per kg of the patient's body weight in a single dose. In the case of transdermal or transmucosal administration, about 1 μg to 50 mg/cm² can be administered, and about 2.5 μg to 10 mg/cm² can be preferably administered.

Hereafter, the present invention is described in greater detail with reference to the following examples, although the technical scope of the present invention is not limited thereto.

### EXAMPLES

### Example 1

20 mg of acid-treated gelatin, 2 mg of daichitosan, 10 mg of a transglutaminase preparation (Activa TG-S, Ajinomoto Co.), 0.4 mg of the model active substance having the structure given below, and 1.79 ml of ion-exchanged water were mixed. The resulting solution (1 ml) was injected into 10 ml of ethanol using a microsyringe with agitation at a preset external temperature of 40° C. at 800 rpm. The resulting dispersion was allowed to stand at a preset external temperature of 55° C. for 5 hours. Thus, crosslinked acid-treated gelatin nanoparticles were obtained. The average particle diameter of such particles was determined to be 85 nm as a result of measurement using a light scattering photometer (DLS-7000, Otsuka Denshi Co., Inc.).

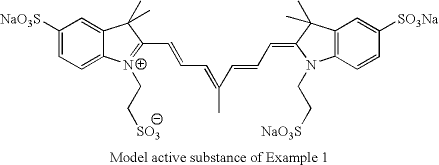

### Example 2

20 mg of albumin, 2 mg of chondroitin sulfate C, 10 mg of a transglutaminase preparation (Activa TG-S, Ajinomoto Co.), 0.4 mg of adriamycin, and 1.79 ml of ion-exchanged water were mixed. The resulting solution (1 ml) was injected into 10 ml of ethanol using a microsyringe with agitation at a preset external temperature of 40° C. at 800 rpm. The resulting dispersion was allowed to stand at a preset external temperature of 55° C. for 5 hours. Thus, crosslinked albumin nanoparticles were obtained. The average particle diameter of such particles was determined to be 30 nm as a result of measurement using a light scattering photometer (DLS-7000, Otsuka Denshi Co., Inc.).

### Example 3

20 mg of the acid-treated gelatin, 10 mg of a transglutaminase preparation (Activa TG-S, Ajinomoto Co.), 0.4 mg of arbutin, and 1.79 ml of ion-exchanged water were mixed. The resulting solution (1 ml) was injected into 10 ml of ethanol that comprises 2 mg of lecithin dissolved therein using a microsyringe with agitation at a preset external temperature of 40° C. at 800 rpm. The resulting dispersion was allowed to stand at a preset external temperature of 55° C. for 5 hours. Thus, crosslinked acid-treated gelatin nanoparticles were obtained. The average particle diameter of such particles was determined to be 90 nm as a result of measurement using a light scattering photometer (DLS-7000, Otsuka Denshi Co., Inc.).

### Example 4

The AquaCollagen® (20 mg), 2 mg of dextran sulfate, 10 mg of a transglutaminase preparation (Activa TG-S, Ajinomoto Co.), 0.4 mg of adriamycin, and 1.79 ml of ion-exchanged water were mixed. The resulting solution (1 ml) was injected into 10 ml of ethanol using a microsyringe with agitation at a preset external temperature of 40° C. at 800 rpm. The resulting dispersion was allowed to stand at a preset external temperature of 55° C. for 5 hours. Thus, crosslinked AquaCollagen nanoparticles were obtained. The average particle diameter of such particles was determined to be 110 nm as a result of measurement using a light scattering photometer (DLS-7000, Otsuka Denshi Co., Inc.).

### Example 5

25 ml of acetone was slowly added to 25 ml of an aqueous solution of 5% gelatin for precipitation. The supernatant was discarded, the precipitate was dissolved in water again, and 2 mg of polylysine, 0.4 mg of the model active substance, and 10 mg of a transglutaminase preparation (Activa TG-S, Ajinomoto Co.) were added thereto. Then, the pH level of the resultant was adjusted to 2.5 for insolubilization. The resulting dispersion was allowed to stand at a preset external temperature of 55° C. for 5 hours. Thus, crosslinked acid-treated gelatin nanoparticles were obtained.

### Example 6

100 mg of acid-treated gelatin was dissolved in 10 ml of ion-exchanged water with heating, hydrochloric acid was added thereto to adjust a pH level to 2.5. Then, 50 mg of a transglutaminase preparation (Activa TG-S, Ajinomoto Co.) and 0.4 mg of arbutin were added. 16 ml of acetone was added dropwise to this solution with agitation, and the mixture was diluted with 230 ml of ethanol. Thus, acid-treated gelatin nanoparticles were obtained. The average particle diameter of such particles was determined to be 90 nm as a result of measurement using a light scattering photometer (DLS-7000, Otsuka Denshi Co., Inc.). A phosphate buffer (10 ml, pH 7) was added dropwise, and the resultant was then subjected to crosslinking at a preset external temperature of 55° C. for 5 hours.

### Example 7

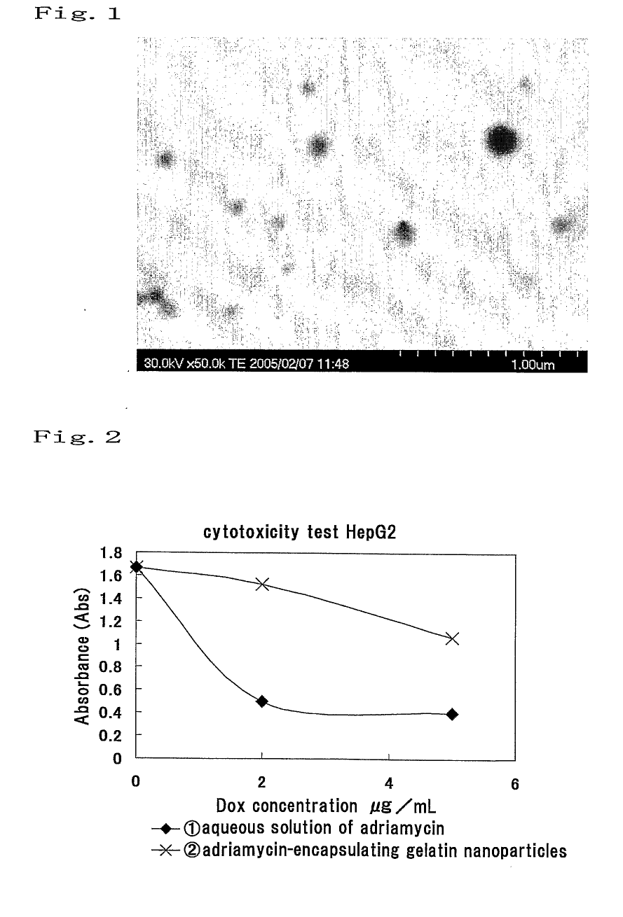

The acid-treated gelatin (10 mg), 5 mg of a transglutaminase preparation (Activa TG-S, Ajinomoto Co.), and 1 ml of ion-exchanged water were mixed. The resulting solution (1 ml) was injected into 10 ml of ethanol using a microsyringe with agitation at a preset external temperature of 40° C. at 800 rpm. The resulting dispersion was allowed to stand at a preset external temperature of 55° C. for 5 hours. Thus, crosslinked and acid-treated gelatin nanoparticles were obtained. The SEM photograph of the particles were taken (FIG. 1).

### Example 8

The acid-treated gelatin (10 mg), 1 mg of chondroitin sulfate C, 5 mg of a transglutaminase preparation (Activa TG-S, Ajinomoto Co.), 0.4 mg of adriamycin (doxorubicin hydrochloride, Wako Pure Chemical Industries, Ltd.), and 1 ml of ion-exchanged water were mixed. The resulting solution (1 ml) was injected into 10 ml of ethanol using a microsyringe with agitation at a preset external temperature of 40° C. at 800 rpm. The resulting dispersion was allowed to stand at a preset external temperature of 55° C. for 5 hours. Thus, crosslinked gelatin nanoparticles were obtained. The average particle diameter of the particles was determined to be 70 nm as a result of measurement using a light scattering photometer (DLS-7000, Otsuka Denshi Co., Inc.). The dispersion of the nanoparticles was subjected to centrifugation, the ethanol supernatant was discarded, and physiological saline was added for redispersion so as to adjust the adriamycin concentration to 200 μg/ml. The adriamycin amount was determined based on the absorption spectrum (Abs. 480 nm). The average particle diameter after the redispersion was determined to be 174 nm as a result of measurement using a light scattering photometer (DLS-7000, Otsuka Denshi Co., Inc.). Since the aforementioned particle size measurement using a light scattering photometer could be performed in a water midium, it was verified that gelatin nanoparticles in which enzyme crosslinking reactions proceeded, which would insolubilize the nanoparticles in water, had been produced.

### Example 9

To a microplate onto which 100 μl each of HepG2 cell-containing solutions had been seeded at a cell density of 20×10³ cells/well, an aqueous solution comprising 2 μg/ml or 5 μg/ml adriamycin and the adriamycin-encapsulating gelatin nanoparticles prepared in Example 8 were added. After culture was conducted for 72 hours, the medium was washed twice. The Cell Counting Kit-8 (Dojin Kagaku) was added in amounts of 10 μl each, color-developing reaction was carried out for 2.5 hours, and the absorption was measured (FIG. 2). Encapsulation of adriamycin in nanoparticles reduced adriamycin toxicity.

### Examples 10

By using a recombinant gelatin (100 kD, FibroGen) instead of the acid-treated gelatin in the above Examples, the same good results were obtained.

### INDUSTRIAL APPLICABILITY

The protein nanoparticle of the present invention is produced from highly biocompatible protein without the use of a surfactant or synthetic polymer, and thus are safe on organisms. In the protein nanoparticle of the present invention, crosslinking is caused by an enzyme without the use of a synthetic chemical crosslinking agent. Thus, the protein nanoparticle of the present invention is highly safe on organisms. In particular, transglutaminase (TG) that can be used in the present invention is a protein that is present in a human body, and thus, the protein nanoparticle of the present invention is highly safe on organisms. Further, microorganism-derived TG is used for protein crosslinking of food, and thus, the protein nanoparticle of the present invention is highly safe as DDS preparations for oral or transdermal administration.

1. A protein nanoparticle which is obtained by enzymatic crosslinking during and/or after the formation of protein nanoparticle.

2. The protein nanoparticle of claim 1 wherein enzymatic crosslinking is performed with the addition of crosslinking enzymes in a weight that is 0.1% to 100% of the protein weight.

3. The protein nanoparticle of claim 1 wherein the enzyme used for crosslinking is transglutaminase.

4. The protein nanoparticle of claim 1 wherein enzymatic crosslinking is carried out in an organic solvent.

5. The protein nanoparticle of claim 1 which further comprises at least one active ingredient.

6. The protein nanoparticle of claim 5 which comprises the active ingredient in a weight that is 0. 1% to 100% of the protein weight.

7. The protein nanoparticle of claim 5 wherein the active ingredient is an ingredient for a cosmetic, functional food, or pharmaceutical product.

8. The protein nanoparticle of claim 7 wherein the ingredient of a cosmetic product is a moisturizer, skin-whitening agent, hair restoration tonics, hormone drugs or antiaging agent, the ingredient of a functional food is a vitamin or antioxidant, and the ingredient of a pharmaceutical product is an anticancer agent, antiallergic agent, antithrombotic agent, immunosuppressive agent, therapeutic agent for skin diseases, antifungal agent, nucleic acid medication or antiinflammatory agent.

9. The protein nanoparticle of claim 1 wherein the average particle size is between 10 nm and 1,000 nm.

10. The protein nanoparticle of claim 1 wherein the protein has a lysine residue and a glutarnine residue.

11. The protein nanoparticle of claim 1 wherein the protein is at least one selected from the group consisting of collagen, gelatin, albumin, casein, transferrin, globulin, fibroin, fibrin, laminin, fibronectin, and vitronectin.

12. The protein nanoparticle of claim 1, wherein the protein is one which is derived from bovine, swine or fish, or a recombinant protein.

13. The protein nanoparticle of claim 1 wherein the protein is an acid-treated gelatin.

14. The protein nanoparticle of claim 1 wherein a phospholipid is added in a weight that is 0.1% to 100% of the protein weight.

15. The protein nanoparticle of claim 1 wherein a cationic or anionic polysaccharide is added in a weight that is 0.1% to 100% of the protein weight.

16. The protein nanoparticle of claim 1 wherein a cationic or anionic protein is added in a weight that is 0.1% to 100% of the protein weight.

17. A drug delivery agent which comprises the protein nanoparticle of claim 1.

18. The drug delivery agent of claim 17 which is used as transdermal absorbents, topical therapeutic agents, oral therapeutic agents, intradermal injections, hypodermic injections, intramuscular injections, intravenous injections, cosmetic products, functional foods, or supplements.

19. The drug delivery agent of claim 17 which comprises an additive.

20. The drug delivery agent of claim 19 wherein the additive is at least one member selected from among moistening agents, softening agents, transdermal absorption promoters, soothing agents, antiseptic agents, antioxidants, pigments, thickeners, aroma chemicals, and pH adjusters.

21. A method for producing a protein nanoparticle which comprises performing enzymatic crosslinking during and/or after the formation of protein nanoparticle.

22. A method for producing a protein nanoparticle which comprises performing enzymatic crosslinking during and/or after the formation of the protein nanoparticle in an organic solvent.
